(19) United States
(12) Patent Application Publication
(10) Pub. No.: US 2022/0315814 A1
(43) Pub. Date: Oct. 6, 2022

(54) ADHESIVE FOR ENDOSCOPE, CURED PRODUCT THEREOF, ENDOSCOPE, AND METHOD FOR PRODUCING THE SAME

(71) Applicant: FUJIFILM Corporation, Tokyo (JP)

(72) Inventors: Kazushi Furukawa, Ashigarakami-gun (JP); Yoshihiro Nakai, Ashigarakami-gun (JP); Shigeki Uehira, Ashigarakami-gun (JP)

(73) Assignee: FUJIFILM Corporation, Tokyo (JP)

(21) Appl. No.: 17/840,266

(22) Filed: Jun. 14, 2022

Related U.S. Application Data
(63) Continuation of application No. PCT/JP2020/044612, filed on Dec. 1, 2020.

(30) Foreign Application Priority Data
Dec. 24, 2019 (JP) 2019-232658

(51) Int. Cl. C09J 163/04; C08G 59/24; C08G 59/40; C08G 59/50; C08K 3/08; A61B 1/005

(52) U.S. Cl. CPC C09J 163/04; C08G 59/245; C08G 59/4021; C08G 59/4064; C08G 59/4071; C08G 59/502; C08K 3/08; A61B 1/005; C08K 2003/0837; C08K 2003/0875; C08K 2003/0812; C08K 2003/0818; C09J 2301/30; C09J 2463/00

(57) ABSTRACT

An adhesive for an endoscope, the adhesive including (A) an epoxy resin including at least one epoxy resin of a bisphenol A epoxy resin, a bisphenol F epoxy resin, or a phenol novolac epoxy resin, (B) a curing component including at least one of a phosphorus-containing compound, a polythiol compound, a dicyandiamide compound, a phenol compound, or a polyether-polyamine compound, and (C) an inorganic amphoteric ion exchanger; and a cured product of the adhesive. An endoscope including the cured product fixed, and a method for producing the endoscope.

### CROSS-REFERENCE TO RELATED APPLICATIONS

This application is a Continuation of PCT International Application No. PCT/JP2020/044612 filed on Dec. 1, 2020, which claims priority under 35 U.S.C. § 119 (a) to Japanese Patent Application No. 2019-232658 filed in Japan on Dec. 24, 2019. Each of the above applications is hereby expressly incorporated by reference, in its entirety, into the present application.

### BACKGROUND OF THE INVENTION

### 1. Field of the Invention

The present invention relates to an adhesive for an endoscope, a cured product of the adhesive, an endoscope, and a method for producing the endoscope.

### 2. Description of the Related Art

Endoscopes for observing the body cavity of the human body are repeatedly used. For this reason, in such an endoscope, the flexible tube forming the insertion section is, after each use, washed or disinfected using a chemical. In particular, in the cases of insertion into highly susceptible regions such as bronchi, cleanliness of the sterilization grade, which is above the disinfection grade, is required. Thus, endoscopes are required to have high durability for resisting even a repeat of sterilization treatment using hydrogen peroxide plasma or ethylene oxide gas, for example.

Endoscopes are inserted through, for example, the oral cavity or the nasal cavity, into the body cavity. In order to lessen foreign body sensation and pain of the patient during insertion, the insertion sections of endoscopes desirably have reduced diameters. Thus, members constituting the insertion sections are joined together using, instead of bulky members such as screws and bolts, mainly adhesives.

Among adhesives, epoxy adhesives have high usability, and their cured products are excellent in adhesiveness, electrical characteristics, heat resistance, and moisture resistance, for example. For this reason, epoxy adhesives are used in various fields, and use of epoxy adhesives for fixing constituent members of endoscopes has been studied.

For example, WO2017/204012A states that an adhesive composition containing an epoxy resin selected from the group consisting of a bisphenol A epoxy resin, a bisphenol F epoxy resin, and a phenol novolac epoxy resin, an amine curing agent, and an inorganic amphoteric ion exchanger is used for connecting together constituent members of an endoscope, and states that an adhesive layer provided by curing this adhesive composition is less likely to undergo deterioration of the appearance even in the case of being subjected to sterilization treatment using hydrogen peroxide plasma. In addition, JP2015-202212A states that a resin composition containing an epoxy resin selected from the group consisting of a bisphenol A epoxy resin, a bisphenol F epoxy resin, a phenol novolac epoxy resin, and epoxy-modified silicone, a curing agent selected from the group consisting of an amine curing agent, a polyamide resin, an imidazole, and an acid anhydride, and an ion exchanger, and including modified silicone in at least one of the epoxy resin or the curing agent is used for a backing material for ultrasonic transducers of ultrasonic endoscopes. JP2015-202212A states that a cured product provided by curing the resin composition can maintain high storage elastic modulus even in the case of being subjected to sterilization treatment using hydrogen peroxide plasma.

### SUMMARY OF THE INVENTION

As described above, for endoscopes, in order to fix their constituent members, epoxy adhesives are frequently used. However, like endoscopes, devices that are subjected to high-temperature washing and strong sterilization treatment and that are produced using existing epoxy adhesives for fixing members, tend to undergo degradation of performance of the devices as a result of repeated use, and hence do not sufficiently satisfy the need.

As described above, WO2017/204012A and JP2015-202212A describe techniques of improving the durability (against sterilization treatment using hydrogen peroxide plasma) of an adhesive layer and a cured product using epoxy adhesives. However, the ethylene oxide gas widely used for sterilization treatment for endoscopes is different from hydrogen peroxide plasma in the compound or ion contributing to the sterilization action and also in the chemical reaction; thus, durability against a repeat of sterilization treatment using ethylene oxide gas needs to be separately studied.

In the technique described in WO2017/204012A, the epoxy adhesive is caused to react in a relatively-high-temperature region (60 to 135° C.) to sufficiently increase the curing speed and the curing ratio, to achieve the above-described sterilization durability.

However, during production of endoscopes, which are precision medical devices, the curing reaction of the epoxy adhesive in the high temperature region may cause failures of the constituent members (precision devices). Thus, adhesives used for producing endoscopes need to have a property of being rapidly cured even in a lower-temperature region.

Objects of the present invention are to provide an adhesive that is an adhesive for an endoscope, the adhesive being suitable for fixing constituent members of endoscopes, that can be rapidly cured even in a low-temperature region, and that provides, as a result of the curing reaction, a cured product having high durability against a repeat of ethylene oxide gas sterilization treatment, which is a widely used sterilization process for endoscopes etc., and a cured product of the adhesive. Other objects of the present invention are to provide an endoscope that is less likely to undergo degradation of the performance even in the case of being subjected to a repeat of ethylene oxide gas sterilization treatment, and a method for producing the endoscope.

In order to achieve the above-described objects, the inventors of the present invention performed thorough studies and, as a result, have found that, in an epoxy adhesive in which a combined use of an epoxy resin serving as the base resin, a specific curing component, and an inorganic amphoteric ion exchanger, the inorganic amphoteric ion exchanger exerts strongly a curing acceleration action to, for example, improve the efficiency of the action of the curing component on the epoxy resin, to achieve rapid curing of the adhesive even in a low-temperature region, and the cured product is less likely to deteriorate even in the case of being subjected to a repeat of sterilization treatment using ethylene oxide gas. On the basis of such findings, they performed further studies and have accomplished the present invention.

The above-described objects have been achieved by the following means.

<1>

An adhesive for an endoscope, the adhesive including:

(A) an epoxy resin including at least one epoxy resin of a bisphenol A epoxy resin, a bisphenol F epoxy resin, or a phenol novolac epoxy resin;

(B) a curing component including at least one of a phosphorus-containing compound, a polythiol compound, a dicyandiamide compound, a phenol compound, or a polyether-polyamine compound; and

(C) an inorganic amphoteric ion exchanger.

<2>

The adhesive for an endoscope according to <1>, wherein the curing component includes at least one of a polythiol compound or a polyether-polyamine compound.

<3>

The adhesive for an endoscope according to <1> or <2>, wherein the inorganic amphoteric ion exchanger is an inorganic compound including at least one species of a bismuth atom, an antimony atom, a zirconium atom, a magnesium atom, or an aluminum atom.

<4>

The adhesive for an endoscope according to any one of <1> to <3>, wherein a content of the inorganic amphoteric ion exchanger relative to 100 parts by mass of the epoxy resin is 1 part by mass or more and 10 parts by mass or less.

<5>

A cured product provided by curing the adhesive for an endoscope according to any one of <1> to <4>.

<6>

An endoscope including a member fixed using the cured product according to <5>.

<7>

A method for producing an endoscope, the method including fixing a member using the adhesive for an endoscope according to any one of <1> to <4>.

In the descriptions of the present invention, when there are a plurality of substituents, linking groups, or the like (hereafter, referred to as substituents etc.) denoted by specific symbols or formulas, or a plurality of substituents etc. are simultaneously defined, the substituents etc. may be the same or different unless otherwise specified. The same applies to the definitions of numbers in substituents etc. When a plurality of substituents etc. are present close to each other (in particular, adjacent to each other), they may be linked together to form a ring unless otherwise specified. In addition, a ring such as an aliphatic ring, an aromatic ring, or a hetero ring may be further fused to form a fused ring unless otherwise specified.

In the descriptions of the present invention, for groups described as examples of substituents, “group” is used in a meaning of encompassing both of the unsubstituted form and forms having a substituent. For example, “alkyl group” means an alkyl group that may have a substituent. When the number of carbon atoms of a group is specified, the number of carbon atoms of the group means the total number of carbon atoms including the substituents unless otherwise specified.

In the descriptions of the present invention, compounds that are not clearly described in terms of being substituted or unsubstituted are intended that they may have appropriate substituents unless the present invention is hindered from providing advantages.

In the descriptions of the present invention, “a value ‘to’ another value” used is intended to include the value and the other value respectively as the lower-limit value and the upper-limit value.

An adhesive for an endoscope according to the present invention can be rapidly cured even in a low-temperature region; a cured product obtained by this curing reaction has high durability against a repeat of ethylene oxide gas sterilization treatment. A cured product according to the present invention has high durability against a repeat of ethylene oxide gas sterilization treatment. Thus, an endoscope according to the present invention, the endoscope having this cured product as a fixing member for a constituent member, is less likely to undergo degradation of performance even in the case of being subjected to a repeat of ethylene oxide gas sterilization treatment. A method for producing an endoscope according to the present invention can provide an endoscope that is less likely to undergo degradation of performance even in the case of being subjected to a repeat of ethylene oxide gas sterilization treatment.

### BRIEF DESCRIPTION OF THE DRAWINGS

FIG. 1 is an external view illustrating the configuration of an endoscope according to an embodiment of the present invention;

FIG. 2 is a partial sectional view illustrating the configuration of the insertion section of the endoscope in FIG. 1;

FIG. 3 is an external perspective view illustrating the distal-end portion of the insertion section; and

FIG. 4 is a cutaway partial sectional view illustrating the distal-end portion in which the sections of the lenses and the prism are not hatched.

### DESCRIPTION OF THE PREFERRED EMBODIMENTS

### Adhesive for Endoscope

Preferred embodiments of an adhesive for an endoscope according to the present invention will be described.

An adhesive for an endoscope according to the present invention (hereafter, also referred to as “adhesive according to the present invention”) includes (A) an epoxy resin, (B) a curing component, and (C) an inorganic amphoteric ion exchanger, wherein the epoxy resin includes at least one of a bisphenol A epoxy resin, a bisphenol F epoxy resin, or a phenol novolac epoxy resin.

The (A) epoxy resin is the base resin of the adhesive. The (B) curing component is a component that reacts with the (A) epoxy resin to cure the adhesive. The (B) curing component used in the present invention includes at least one of a phosphorus-containing compound, a polythiol compound, a dicyandiamide compound, a phenol compound, or a polyether-polyamine compound. The (C) inorganic amphoteric ion exchanger serves as a curing acceleration component accelerating the curing reaction.

In the following descriptions, the (A) epoxy resin, the (B) curing component, and the (C) inorganic amphoteric ion exchanger may also be simply referred to as respectively Component (A), Component (B), and Component (C).

An adhesive according to the present invention, as long as it includes the above-described components, is not limited in terms of form. For example, an adhesive for an endoscope according to the present invention may have a form containing a mixture of Components (A) to (C) above (one-component type), or may include Components (A) to (C) above such that one or more components among Components (A) to (C) are separated from the other component (two-component type). Alternatively, an adhesive for an endoscope according to the present invention may include Components (A) to (C) such that Components (A) to (C) are separated from each other (three-component type). All these forms fall within the scope of an adhesive according to the present invention.

In this Specification, descriptions of the contents of components in adhesives or definitions of, in the present invention, the contents of components in adhesives are intended that, in the cases of the form of the two-component type or the three-component type, Components (A) to (C) are mixed at the time of use such that the components satisfy the desired contents in the mixtures. In other words, in the cases of the form of the two-component type or the three-component type, in a state where the components are separated, the contents of Components (A) to (C) do not necessarily satisfy the contents described in this Specification or the contents defined in the present invention. Stated another way, the form of the two-component type or the three-component type satisfies, at the time when Components (A) to (C) are mixed together for use, the contents described in this Specification or the contents defined in the present invention.

When an adhesive for an endoscope according to the present invention is of the one-component type or even the two-component type or the like and includes a mixture of components that react with each other (for example, a mixture of an epoxy resin and a curing component), in order to prevent or sufficiently suppress the reaction between the components to keep a state in which the components are maintained with stability, the adhesive is preferably stored at such a low temperature that the reaction substantially does not occur. For example, the storage can be performed at −20° C. or less, preferably −30° C. or less, more preferably −40° C. or less, still more preferably −50° C. or less. The storage can be performed under light-tight conditions as needed.

An adhesive according to the present invention may include, unless the present invention is hindered from providing advantages, for example, a solvent, a plasticizer, an adhesiveness improver (such as a silane coupling agent), a surfactant, a coloring agent (such as a pigment or a dye), an anti-weathering agent, an antioxidant, a heat stabilizer, a lubricant, an antistatic agent, a brightening agent, a release agent, a conductive agent, a viscosity modifier, a filler (such as silica or calcium carbonate), a thixotropy imparting agent, a diluent, and a flame retardant.

An adhesive according to the present invention can be rapidly cured in the case of causing the curing reaction even in a low-temperature region (for example, less than 60° C., preferably 0 to 50° C.), and the resultant cured product has high durability against a repeat of sterilization treatment using ethylene oxide gas, for example. The reason for this is not clarified, but is inferred as follows.

Specifically, the low-temperature curing reactivity is inferentially provided partly because, in an adhesive according to the present invention, the inorganic amphoteric ion exchanger interacts, via its metallic atom, with oxygen atoms of the epoxy resin, and similarly interacts with a heteroatom etc. of the curing component (for example, P, S, CN, or 0 in the curing component). Such interactions inferentially cause the epoxy resin and the curing component to become closer to each other, to cause, even in a low-temperature region, the curing reaction to proceed rapidly and sufficiently.

The improvement in the durability against ethylene oxide gas sterilization treatment (suppression of decrease in mechanical strength) is also inferentially attributable to an ionic interaction via a metallic atom of the inorganic amphoteric ion exchanger. For example, inferentially, in a cured product according to the present invention, a lone electron pair of the oxygen atom of ethylene oxide ionically interacts with a metallic atom of the inorganic amphoteric ion exchanger, so that ethylene oxide is trapped; as described above, the metallic atom also interacts with a heteroatom in the curing component, so that a bulky structure is formed using the curing component. As a result, inferentially, migration of trapped ethylene oxide in the cured product can be effectively suppressed, so that ethylene oxide molecules become less likely to come into contact with each other, generation of liquid components such as polyethylene glycol is suppressed, and the mechanical strength of the cured product becomes less likely to decrease.

An adhesive according to the present invention is used to fix various constituent members of an endoscope (endoscope constituent members). Specifically, an adhesive according to the present invention is used to bond an endoscope constituent member to another endoscope constituent member, to thereby fix the endoscope constituent member to the other endoscope constituent member. The adhesive having been used for fixing the endoscope constituent member turns into a cured product to form a bonding portion of the endoscope.

The member fixed using an adhesive according to the present invention is not particularly limited, and preferred examples include metal members, glass members, and resin members. Such an endoscope constituent member is “fixed” by bonding the endoscope constituent member to another constituent member (support member) of the endoscope. Incidentally, the support member may be, for example, the tube wall of the endoscope or an immovable member fixed on the tube wall or the like, or may be a member that is movable to another relative position within the endoscope, such as the tube. In the present invention, the term “fix” is used with meanings including filling, with a cured product of an adhesive, the gap between an endoscope constituent member and a support member into which the endoscope constituent member is incorporated, namely, sealing.

Hereinafter, constituent components of an adhesive according to the present invention will be described.

### (A) Epoxy Resin

An adhesive according to the present invention includes an epoxy resin; this epoxy resin includes at least one of a bisphenol A epoxy resin, a bisphenol F epoxy resin, or a phenol novolac epoxy resin. An adhesive according to the present invention may include one or two or more epoxy resins selected from the group consisting of a bisphenol A epoxy resin, a bisphenol F epoxy resin, and a phenol novolac epoxy resin.

Relative to the total amount of epoxy resin included in an adhesive according to the present invention, the percentage of the total amount of the bisphenol A epoxy resin, the bisphenol F epoxy resin, and the phenol novolac epoxy resin is preferably 70 mass % or more, preferably 80 mass % or more, more preferably 90 mass % or more. More preferably, the epoxy resin included in an adhesive according to the present invention is at least one of a bisphenol A epoxy resin, a bisphenol F epoxy resin, or a phenol novolac epoxy resin.

The epoxy resin included in an adhesive according to the present invention preferably has an epoxy equivalent of 10 to 1000, more preferably 50 to 500, still more preferably 80 to 400, particularly preferably 100 to 300. The epoxy resin included in an adhesive according to the present invention ordinarily has two or more epoxy groups in a single molecule.

The epoxy equivalent is a value obtained by dividing the mass-average molecular weight of an epoxy compound by the number of moles of the epoxy groups of the epoxy compound.

The bisphenol A epoxy resin usable for an adhesive according to the present invention is not particularly limited, and such resins ordinarily used as the base resins of epoxy adhesives can be widely employed. Preferred specific examples include bisphenol A diglycidyl ethers (jER 825, jER 828, and jER 834 (all of which are trade names), manufactured by Mitsubishi Chemical Corporation) and a bisphenol A propoxylate diglycidyl ether (manufactured by Sigma-Aldrich Corporation).

The bisphenol F epoxy resin usable for an adhesive according to the present invention is not particularly limited, and such resins ordinarily used as the base resins of epoxy adhesives can be widely employed. Preferred specific examples include a bisphenol F diglycidyl ether (trade name: EPICLON 830, manufactured by DIC Corporation) and 4,4′-methylenebis(N,N-diglycidylaniline).

The phenol novolac epoxy resin usable for an adhesive according to the present invention is not particularly limited, and such resins ordinarily used as the base resins of epoxy adhesives can be widely employed. Such a phenol novolac epoxy resin is, for example, commercially available as product number: 406775 from Sigma-Aldrich Corporation.

The content of the epoxy resin included in an adhesive according to the present invention is preferably 5 mass % or more, more preferably 10 mass % or more, more preferably 20 mass % or more, more preferably 30 mass % or more, more preferably 40 mass % or more, still more preferably 50 mass % or more. The content of the epoxy resin is preferably 99 mass % or less, more preferably 98 mass % or less, more preferably 97 mass % or less, still more preferably 96 mass % or less.

### (B) Curing Component

An adhesive according to the present invention includes, as Component (B), at least one of a phosphorus-containing compound, a polythiol compound, a dicyandiamide compound, a phenol compound, or a polyether-polyamine compound. In an adhesive according to the present invention, the phosphorus-containing compound, the polythiol compound, the dicyandiamide compound, the phenol compound, and the polyether-polyamine compound may be used alone or in combination of two or more thereof.

An adhesive according to the present invention, from the viewpoint of being rapidly curable even in a low-temperature region and further improving the sterilization durability, preferably includes, as Component (B), at least one of a polythiol compound or a polyether-polyamine compound.

### (1) Phosphorus-Containing Compound

As a phosphorus-containing compound used in the present invention, compounds ordinarily used as curing accelerators for epoxy resins can be widely employed. The phosphorus-containing compound may be, for example, a tertiary phosphine compound or a tetra-substituted phosphonium tetra-substituted borate.

The tertiary phosphine compound is, for example, a phosphine compound in which three groups in total among alkyl groups and aryl groups are bonded to the phosphorus atom. Such an alkyl group may be linear, branched, or cyclic, and is preferably an alkyl group having 1 to 20 carbon atoms, more preferably an alkyl group having 1 to 10 carbon atoms, still more preferably an alkyl group having 1 to 5 carbon atoms. Specific examples of the alkyl group include methyl, ethyl, propyl, butyl, t-butyl, and cyclohexyl. Such an aryl group preferably has 6 to 20, more preferably 6 to 10 carbon atoms; the aryl group may be specifically, for example, phenyl or naphthyl, and is preferably phenyl. The aryl group may have a substituent; specific examples of the substituent include the above-described alkyl groups.

The phosphine compound in which three groups in total among alkyl groups and aryl groups are bonded to the phosphorus atom is preferably a triarylphosphine, a trialkylphosphine, or a monoalkyldiarylphosphine.

Specific examples of the triarylphosphine include triphenylphosphine, tris(4-methylphenyl)phosphine, tris(4-ethylphenyl)phosphine, tris(4-propylphenyl)phosphine, tris(4-butylphenyl)phosphine, tris(2,4-dimethylphenyl)phosphine, and tris(2,4,6-trimethylphenyl)phosphine.

Specific examples of the trialkylphosphine include tributylphosphine, trioctylphosphine, tricyclohexylphosphine, and triisobutylphosphine.

Specific examples of the monoalkyldiarylphosphine include methyldiphenylphosphine, ethyldiphenylphosphine, hexyldiphenylphosphine, and cyclohexyldiphenylphosphine.

The tetra-substituted phosphonium tetra-substituted borate may be, for example, a tetraarylphosphonium tetraaryl borate or a tetraalkylphosphonium tetraalkyl borate. As the aryl groups of the tetraarylphosphonium tetraaryl borate and the alkyl groups of the tetraalkylphosphonium tetraalkyl borate may be the alkyl groups and the aryl groups that can be used in the phosphine compound in which three groups in total among alkyl groups and aryl groups are bonded to the phosphorus atom.

Specific examples of the tetraarylphosphonium tetraaryl borate include tetraphenylphosphonium tetraphenyl borate, tetraphenylphosphonium tetra-p-tolyl borate and p-tolyltriphenylphosphonium tetra-p-tolyl borate.

Specific examples of the tetraalkylphosphonium tetraalkyl borate include tetrabutylphosphonium tetrabutyl borate, tri-tert-butylmethylphosphonium tetrabutyl borate, and di-tert-butyldimethylphosphonium tetrabutyl borate.

In an adhesive according to the present invention, the triarylphosphine is preferred; in particular, preferred are triphenylphosphine and tris(4-methylphenyl)phosphine.

### (2) Polythiol Compound

As the polythiol compound used in the present invention, compounds ordinarily used as curing accelerators for epoxy resins can be widely employed. For example, a compound that has at least two moieties represented by General formula (1) below, or at least two moieties represented by General formula (2) below can be employed. Such a polythiol compound that has a structure having 3 to 10 (preferably 3 to 6) moieties represented by General formula (1) below, or 3 to 10 (preferably 3 to 6) moieties represented by General formula (2) below is preferred from the viewpoint that the cured product has increased crosslinking density to have further improved sterilization durability. On the other hand, such a polythiol compound that has a structure having two moieties represented by General formula (1) below, or two moieties represented by General formula (2) below is preferred from the viewpoint that the resultant cured product is relatively flexible.

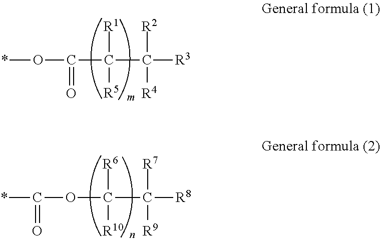

In General formula (1), one of R¹ to R⁵ represents a sulfanyl group (thiol group), and the others each independently represent a hydrogen atom, an alkyl group having 1 to 10 carbon atoms, or an aryl group having 6 to 14 carbon atoms; m represents an integer of 0 to 2. When m is 2, two R¹'s may be the same or different, and two R⁵'s may be the same or different. * denotes the point of attachment in the thiol compound.

In General formula (2), one of R⁶ to R¹⁰ represents a sulfanyl group, and the others each independently represent a hydrogen atom, an alkyl group having 1 to 10 carbon atoms, or an aryl group having 6 to 14 carbon atoms; n represents an integer of 0 to 2. When n is 2, two R⁶'s may be the same or different, and two R¹⁶'s may be the same or different. * denotes the point of attachment in the polythiol compound.

The alkyl group having 1 to 10 carbon atoms may be linear or branched, and examples include methyl, ethyl, propyl, i-propyl, butyl, i-butyl, t-butyl, hexyl, and octyl. Of these, methyl or ethyl is preferred.

Specific examples of the aryl group having 6 to 14 carbon atoms include phenyl and naphthyl.

m is preferably 0 or 1.

n is preferably 0 or 1.

The moiety represented by General formula (1) above is preferably a moiety represented by General formula (3) below.

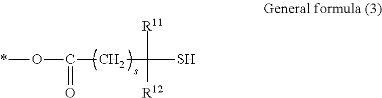

In General formula (3), R¹¹ and R¹² each independently represent a hydrogen atom or an alkyl group having 1 to 10 carbon atoms, and s represents an integer of 0 to 2. * denotes the point of attachment in the polythiol compound.

At least one of R¹¹ or R′² preferably represents an alkyl group having 1 to 10 carbon atoms.

The alkyl groups having 1 to 10 carbon atoms represented by R¹¹ and R′² have the same definitions and preferred examples as the above-described alkyl group that can be employed as R¹ in General formula (1).

s is preferably 0 or 1, more preferably 1.

The polythiol compound is preferably an ester of a compound represented by General formula (4) below and a polyfunctional alcohol.

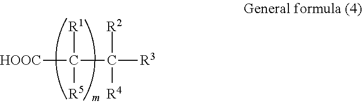

In General formula (4), R¹ to R⁵ and m respectively have the same definitions and preferred examples as R¹ to R⁵ and m in General formula (1) above.

The compound represented by General formula (4) is preferably a compound represented by General formula (5) below.

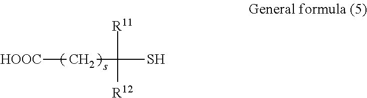

In General formula (5), R¹¹, R¹², and s respectively have the same definitions and preferred examples as R¹¹, R¹², and s in General formula (3) above.

Specific examples of the compound represented by General formula (4) above include 2-mercaptopropionic acid, 3-mercaptopropionic acid, 3-mercaptobutanoic acid, 2-mercaptoisobutanoic acid, 3-mercapto-3-phenylpropionic acid, 3-mercaptoisobutyric acid, 2-mercapto-3-methylbutyric acid, 3-mercapto-3-methylbutyric acid, 3-mercaptovaleric acid, and 3-mercapto-4-methylvaleric acid.

The polyfunctional alcohol is preferably an alcohol having 2 to 10 functionality (polyol having 2 to 10 hydroxy groups), more preferably 2 to 8 functionality, particularly preferably 2 to 6 functionality.

Specific examples of the polyfunctional alcohol include alkylene glycols (the alkylene groups preferably have 2 to 10 carbon atoms, and the alkylene groups may be linear or branched), diethylene glycol, glycerol, dipropylene glycol, trimethylolpropane, pentaerythritol, and dipentaerythritol.

Examples of the alkylene glycols include ethylene glycol, trimethylene glycol, 1,2-propane glycol, 1,2-butanediol, 1,3-butanediol, 2,3-butanediol, and tetramethylene glycol.

Preferred examples of the polyfunctional alcohol include alkylene glycols having an alkylene main chain having 2 carbon atoms, such as ethylene glycol, 1,2-propane glycol, and 1,2-butanediol, trimethylolpropane, and pentaerythritol.

The following are specific examples of the polythiol compound usable in the present invention; however, the present invention is not limited to these.

Specific examples include bis(1-mercaptoethyl) phthalate, bis(2-mercaptopropyl) phthalate, bis(3-mercaptobutyl) phthalate, bis(3-mercaptoisobutyl) phthalate, ethylene glycol bis(3-mercaptopropionate), ethylene glycol bis(3-mercaptobutyrate), propylene glycol bis(3-mercaptobutyrate), diethylene glycol bis(3-mercaptobutyrate), tetraethylene glycol bis(3-mercaptopropionate), butanediol bis(3-mercaptobutyrate), octanediol bis(3-mercaptobutyrate), trimethylolpropane tris(3-mercaptobutyrate), trimethylolpropane tris(3-mercaptopropionate), dipentaerythritol hexakis(3-mercaptopropionate), pentaerythritol tetrakis(3-mercaptopropionate), pentaerythritol tetrakis(3-mercaptobutyrate), dipentaerythritol hexakis(3-mercaptobutyrate), ethylene glycol bis(2-mercaptopropionate), propylene glycol bis(2-mercaptopropionate), diethylene glycol bis(2-mercaptopropionate), butanediol bis(2-mercaptopropionate), octanediol bis(2-mercaptopropionate), trimethylolpropane tris(2-mercaptopropionate), pentaerythritol tetrakis(2-mercaptopropionate), dipentaerythritol hexakis(2-mercaptopropionate), ethylene glycol bis(3-mercaptoisobutyrate), propylene glycol bis(3-mercaptoisobutyrate), diethylene glycol bis(3-mercaptoisobutyrate), butanediol bis(3-mercaptoisobutyrate), octanediol bis(3-mercaptoisobutyrate), trimethylolpropane tris(3-mercaptoisobutyrate), pentaerythritol tetrakis(3-mercaptoisobutyrate), dipentaerythritol hexakis(3-mercaptoisobutyrate), ethylene glycol bis(2-mercaptoisobutyrate), propylene glycol bis(2-mercaptoisobutyrate), diethylene glycol bis(2-mercaptoisobutyrate), butanediol bis(2-mercaptoisobutyrate), octanediol bis(2-mercaptoisobutyrate), trimethylolpropane tris(2-mercaptoisobutyrate), pentaerythritol tetrakis(2-mercaptoisobutyrate), dipentaerythritol hexakis(2-mercaptoisobutyrate), ethylene glycol bis(4-mercaptovalerate), propylene glycol bis(4-mercaptoisovalerate), diethylene glycol bis(4-mercaptovalerate), butanediol bis(4-mercaptovalerate), octanediol bis(4-mercaptovalerate), trimethylolpropane tris(4-mercaptovalerate), pentaerythritol tetrakis(4-mercaptovalerate), dipentaerythritol hexakis(4-mercaptovalerate), ethylene glycol bis(3-mercaptovalerate), propylene glycol bis(3-mercaptovalerate), diethylene glycol bis(3-mercaptovalerate), butanediol bis(3-mercaptovalerate), octanediol bis(3-mercaptovalerate), trimethylolpropane tris(3-mercaptovalerate), pentaerythritol tetrakis(3-mercaptovalerate), dipentaerythritol hexakis(3-mercaptovalerate), 1,4-bis(3-mercaptobutyryloxy)butane, 1,3,5-tris(3-mercaptobutyloxyethyl)-1,3,5-triazine-2,4,6 (1H,3H,5H)-trione, and tris[(3-mercaptopropionyloxy)ethyl] isocyanurate.

From the viewpoint of having less odor, desired viscosities, and high compatibility with the epoxy resin serving as Component (A), and providing the handleability of a mixture obtained by mixing Component (A) and Component (B), the polythiol compound is preferably at least one of 1,4-bis(3-mercaptobutyryloxy)butane, pentaerythritol tetrakis(3-mercaptobutyrate), 1,3,5-tris(3-mercaptobutyloxyethyl)-1,3,5-triazine-2,4,6 (1H,3H,5H)-trione, trimethylolpropane tris(3-mercaptobutyrate), ethylene glycol bis(3-mercaptopropionate), tetraethylene glycol bis(3-mercaptopropionate), trimethylolpropane tris(3-mercaptopropionate), pentaerythritol tetrakis(3-mercaptopropionate), dipentaerythritol hexakis(3-mercaptopropionate), or tris[(3-mercaptopropionyloxy)ethyl] isocyanurate, more preferably at least one of 1,4-bis(3-mercaptobutyryloxy)butane, pentaerythritol tetrakis(3-mercaptobutyrate), 1,3,5-tris(3-mercaptobutyloxyethyl)-1,3,5-triazine-2,4,6(1H,3H,5H)-trione, or trimethylolpropane tris(3-mercaptobutyrate).

The molecular weight of the polythiol compound is not particularly limited, but is, from the viewpoint of handleability in which, for example, it is easily mixed with the epoxy resin serving as Component (A) of an adhesive according to the present invention and is less likely to separate again, and a mixture obtained by mixing Component (A) and Component (B) is less likely to cause sagging or unevenness, preferably 200 to 1,000, more preferably 300 to 800.

In the present invention, the polythiol compound employed may be a commercially available product, and specific examples include 1,4-bis(3-mercaptobutyryloxy)butane (trade name: Karenz MT BD1, manufactured by SHOWA DENKO K. K.), pentaerythritol tetrakis(3-mercaptobutyrate) (trade name: Karenz MT PE1, manufactured by SHOWA DENKO K. K.), 1,3,5-tris(3-mercaptobutyl oxyethyl)-1,3,5-triazine-2,4,6(1H,3H,5H)-trione (trade name: Karenz MT NR1, manufactured by SHOWA DENKO K. K.), and trimethylolpropane tris(3-mercaptobutyrate) (trade name: TPMB, manufactured by SHOWA DENKO K. K.).

### (3) Dicyandiamide Compound

The dicyandiamide compound used in the present invention is a compound that is widely used as a curing accelerator for epoxy resins, has, as the basic skeleton, dicyandiamide, and that exerts a curing acceleration action. Such compounds include, in addition to dicyandiamide ((NH₂)₂C═NCN) itself, compounds having a structure in which the hydrogen atoms of the amino groups of dicyandiamide are partly substituted. As the dicyandiamide compounds, for example, DICY7 and DICY15 (both of which are trade names, manufactured by Mitsubishi Chemical Corporation) are commercially available. Alternatively, in an adhesive according to the present invention, commercially available products manufactured by Tokyo Chemical Industry Co., Ltd. can be used.

### (4) Phenol Compound

The phenol compound used in the present invention is a compound that is widely used as a curing agent for epoxy resins, and that has a phenolic hydroxy group. The term “phenolic hydroxy group” refers to a hydroxy group that is bonded to a ring-forming carbon atom of an aromatic hydrocarbon ring.

The phenol compound used in the present invention is preferably phenol resins; specific examples of the phenol resins include HF-1M, DL-92, MEHC-7841-45, and MEH-7000 manufactured by Meiwa Plastic Industries, Ltd., and TD-2131 and TD-2106 manufactured by DIC Corporation (all of which are trade names).

### (5) Polyether-Polyamine Compound

Such a polyamine compound used in the present invention is a compound that has, in a single molecule, two or more amino groups having an active hydrogen, and that has a polyoxyalkylene group. This polyether-polyamine compound preferably has an unsubstituted amino group (—NH₂), more preferably has two or more unsubstituted amino groups. This polyether-polyamine compound is still more preferably a primary polyamine compound (a polyamine compound in which all the amino groups are unsubstituted amino groups). For an adhesive according to the present invention, polyether-polyamine compounds that exert a curing action in epoxy adhesives can be widely employed.

In a single molecule of the polyether-polyamine compound, the number of amino groups having an active hydrogen is preferably 2 to 10, more preferably 2 to 8, still more preferably 2 to 6, still more preferably 2 to 4, particularly preferably 2 or 3. In particular, at least one selected from the group consisting of diamine compounds and triamine compounds can be suitably used.

The polyether-polyamine compound preferably has an active-hydrogen equivalent (the equivalent of the active hydrogens of amino groups) of 10 to 2000, more preferably 20 to 1000, still more preferably 30 to 900, still more preferably 40 to 800, still more preferably 60 to 700, particularly preferably 65 to 600.

The active-hydrogen equivalent is a value obtained by dividing the molecular weight of the polyether-polyamine compound by the number of moles of active hydrogens of amino groups in the polyamine compound (it means the molecular weight per active hydrogen of amino groups in the polyamine compound).

The polyether-polyamine compound preferably has a number-average molecular weight of 100 to 6000, more preferably 100 to 3000.

The polyether-polyamine compound preferably has a form having a poly(oxyalkylene) structure in which two or more amino groups are bonded to the poly(oxyalkylene) structure via a group selected from the group consisting of an aliphatic hydrocarbon group, a cyclic hydrocarbon group, an aromatic hydrocarbon group, and a heterocyclic group or a group of a combination of the foregoing, more preferably has a form in which two or more amino groups are bonded to the poly(oxyalkylene) structure via an aliphatic hydrocarbon group, an oxygen atom, or a group of a combination of the foregoing.

The aliphatic hydrocarbon group is a di- to tetra-valent aliphatic hydrocarbon group, preferably a di- to tetra-valent saturated aliphatic hydrocarbon group. The aliphatic hydrocarbon group preferably has 2 to 50, more preferably 2 to 30, still more preferably 2 to 10 carbon atoms.

The alkylene group of the poly(oxyalkylene) structure may be a linear alkylene group or an alkylene group having a branch. In the alkylene group of the poly(oxyalkylene) structure, the number of carbon atoms is preferably 1 to 10, more preferably 2 to 6, still more preferably 2 to 4.

The poly(oxyalkylene) structure is more preferably a poly(oxyethylene) group or a poly(oxypropylene) group.

The plurality of alkylene groups constituting the poly(oxyalkylene) structure may be the same or different. The average repeating number of the oxyalkylene groups in the poly(oxyalkylene) structure is preferably 2 to 1000, more preferably 3 to 500. The average repeating number is also preferably 2 to 100, also preferably 2 to 50, also preferably 2 to 35, also preferably 2 to 25. The polyamine compound of Component (B) may have a plurality of polyoxyalkylene structures; these polyoxyalkylene structures may be the same or different.

Specific preferred examples of the polyamine compound usable for the present invention are as follows. The numbers at the parentheses are the average repeating numbers of the repeating units within the parentheses.

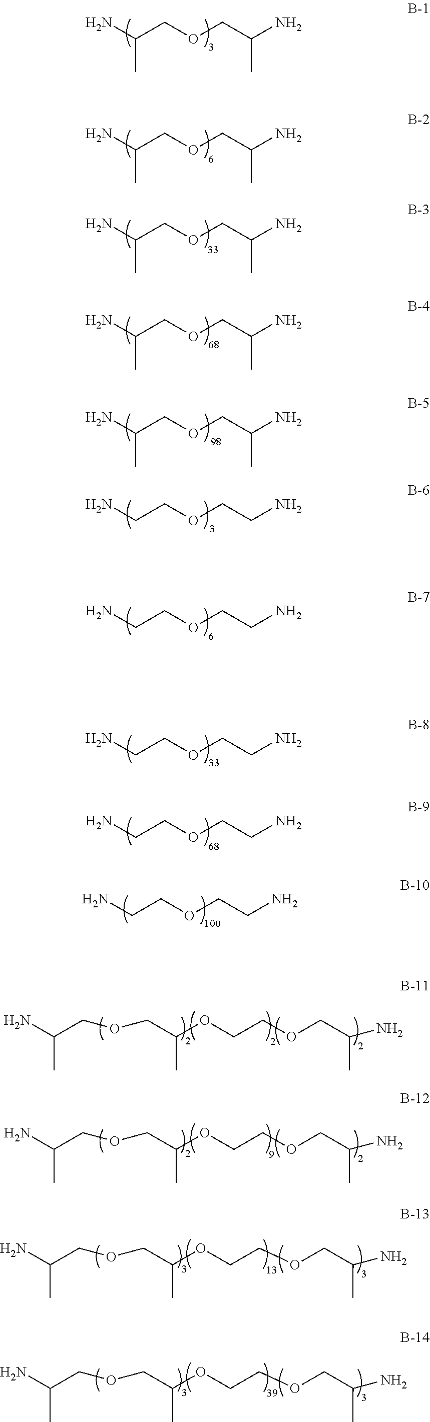

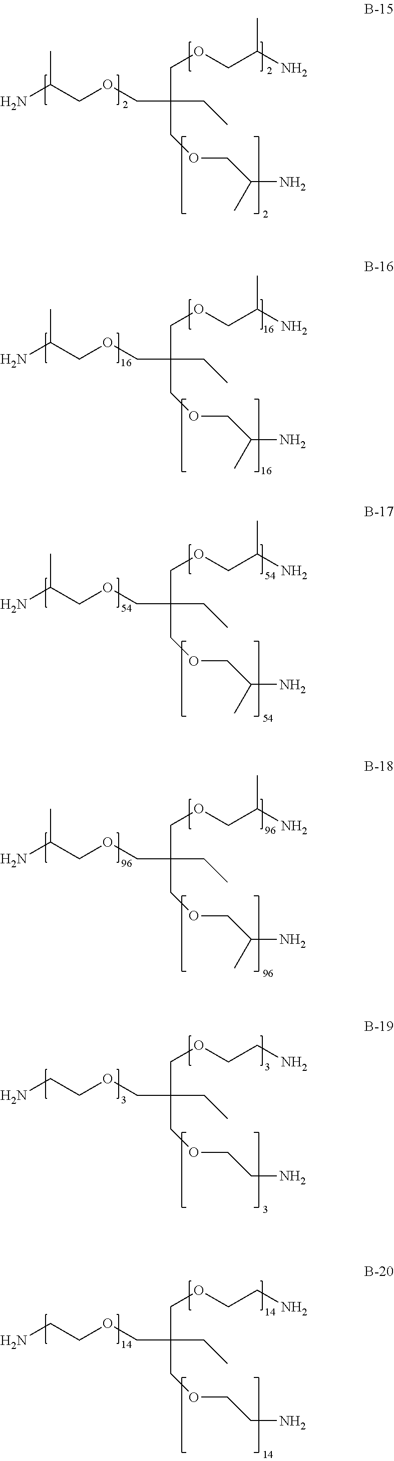

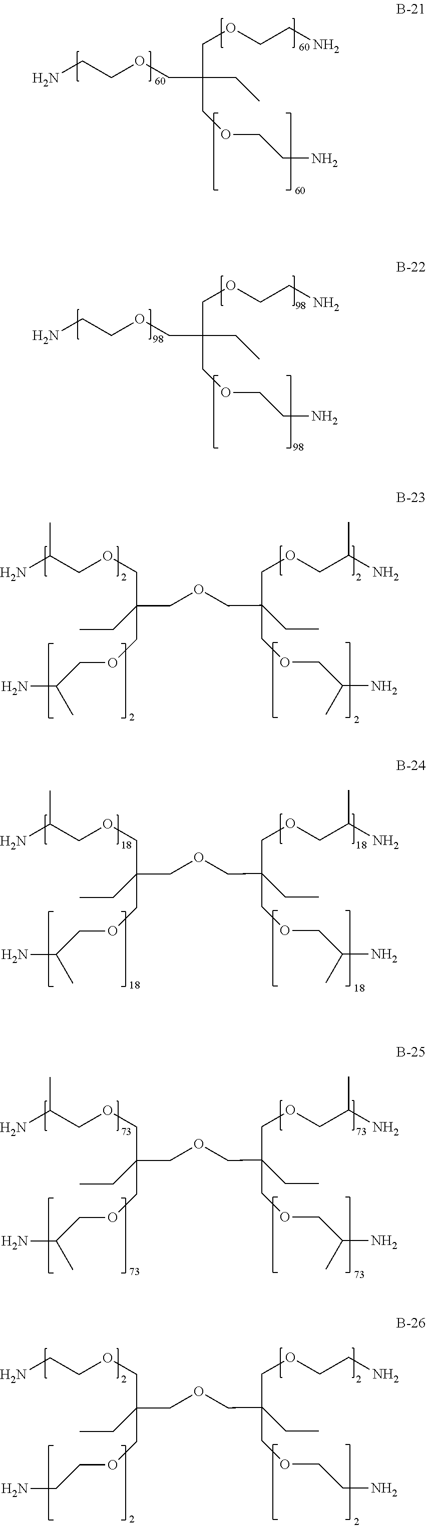

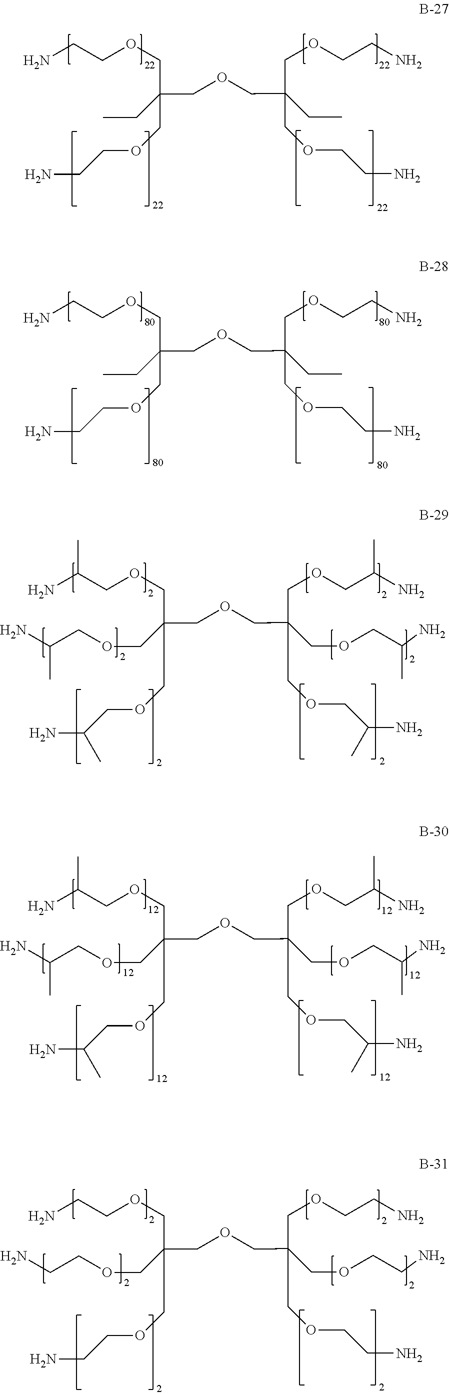

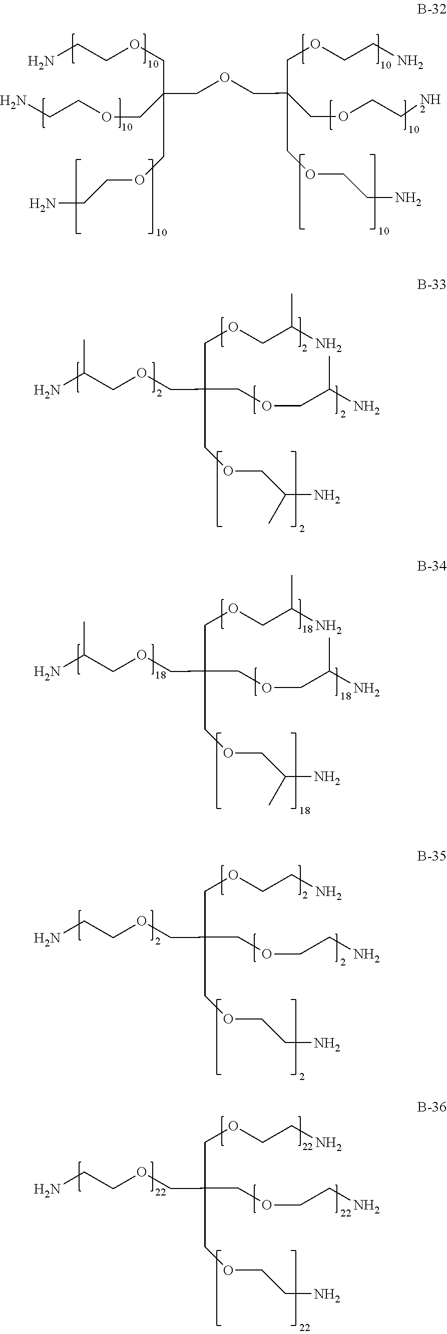

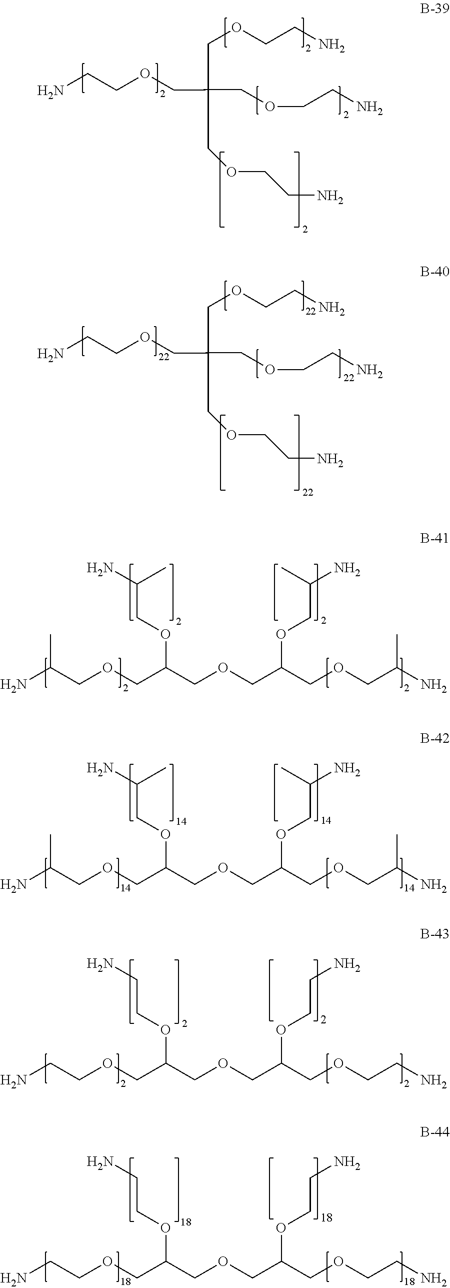

The above-described polyamine compounds can be synthesized in the standard manner. Alternatively, commercially available products may be used.

In Component (B) used in an adhesive according to the present invention, the percentage of a phosphorus-containing compound, a polythiol compound, a dicyandiamide compound, a phenol compound, and a polyether-polyamine compound is preferably 80 mass % or more, more preferably 90 mass % or more. Component (B) is also preferably composed only of one or a combination of two or more selected from the group consisting of a phosphorus-containing compound, a polythiol compound, a dicyandiamide compound, a phenol compound, and a polyether-polyamine compound. When an adhesive according to the present invention includes, as Component (B), a curing component other than a phosphorus-containing compound, a polythiol compound, a dicyandiamide compound, a phenol compound, and a polyether-polyamine compound, the curing component employed can be an imidazole compound, for example.

In an adhesive according to the present invention, the content of Component (B) is not particularly limited, and can be appropriately adjusted in accordance with, for example, the reaction between Component (A) and Component (B).

When Component (B) includes the phosphorus-containing compound, in an adhesive according to the present invention, the content of the phosphorus-containing compound relative to 100 parts by mass of the epoxy resin serving as Component (A) is preferably 0.1 to 10 parts by mass, more preferably 0.3 to 5 parts by mass, still more preferably 0.5 to 4 parts by mass.

When Component (B) includes the polythiol compound, in an adhesive according to the present invention, the content of the polythiol compound can be appropriately set in consideration of active-hydrogen equivalent, for example.

In an adhesive according to the present invention, the content of the polythiol compound relative to 100 parts by mass of the epoxy resin serving as Component (A) is preferably 15 to 100 parts by mass, more preferably 20 to 90 parts by mass, more preferably 35 to 90 parts by mass, still more preferably 50 to 80 parts by mass.

When Component (B) includes the dicyandiamide compound, in an adhesive according to the present invention, the content of the dicyandiamide compound can be appropriately set in consideration of active-hydrogen equivalent, for example.

For example, the content relative to 100 parts by mass of the epoxy resin serving as Component (A) can be set at 1 to 70 parts by mass, and is preferably 10 to 60 parts by mass, more preferably 20 to 65 parts by mass, still more preferably 30 to 50 parts by mass. The ratio of the active-hydrogen equivalent of the dicyandiamide compound to the epoxy equivalent of the epoxy resin serving as Component (A) (active-hydrogen equivalent/epoxy equivalent) is preferably set at 0.3 to 1.0, more preferably 0.4 to 0.7.

When Component (B) includes the phenol compound, in an adhesive according to the present invention, the content of the phenol compound relative to 100 parts by mass of the epoxy resin serving as Component (A) is preferably 3 to 60 parts by mass, more preferably 5 to 50 parts by mass, more preferably 10 to 40 parts by mass, still more preferably 15 to 35 parts by mass.

When Component (B) includes the polyether-polyamine compound, in an adhesive according to the present invention, the content of the polyether-polyamine compound can be appropriately set in consideration of active-hydrogen equivalent, for example.

For example, relative to 100 parts by mass of the epoxy resin serving as Component (A), the content can be set at 10 to 100 parts by mass, and is more preferably 20 to 90 parts by mass, more preferably 30 to 80 parts by mass, still more preferably 40 to 70 parts by mass. The ratio of the active-hydrogen equivalent of the polyether-polyamine compound to the epoxy equivalent of the epoxy resin serving as Component (A) (active-hydrogen equivalent/epoxy equivalent) is preferably set at 0.1 to 1.5, more preferably 0.3 to 1.0, still more preferably 0.5 to 1.0.

### (C) Inorganic Amphoteric Ion Exchanger

An adhesive according to the present invention contains an inorganic amphoteric ion exchanger.

The term “inorganic amphoteric ion exchanger” means a metal-containing inorganic compound that causes an ion exchange phenomenon for both of a cation and an anion. Specifically, an inorganic amphoteric ion exchanger used in the present invention is an inorganic compound that can, upon in contact with an aqueous solution of a salt, release the cation and the anion of the inorganic amphoteric ion exchanger itself into the solution, and that instead incorporate the cation and the anion in the solution into the inorganic amphoteric ion exchanger.

The inorganic amphoteric ion exchanger that can be used for an adhesive according to the present invention is not particularly limited, and is preferably an inorganic compound including at least one species of a bismuth (Bi) atom, an antimony (Sb) atom, a zirconium (Zr) atom, a magnesium (Mg) atom, or an aluminum (Al) atom, more preferably an inorganic compound including two or three species of the atoms.

Examples of the combination of two species of the atoms include a combination of an Sb atom and a Bi atom and a combination of a Zr atom and a Bi atom. Specific examples of the inorganic amphoteric ion exchanger having a combination of an Sb atom and a Bi atom include IXE-600 and IXE-633. Specific examples of the inorganic amphoteric ion exchanger having a combination of a Zr atom and a Bi atom include IXE-6107, IXE-6136, and IXEPLAS-B1 (all of which are trade names (manufactured by TOAGOSEI CO., LTD.)).

The combination of three species of the atoms is, for example, a combination of a Zr atom, a Mg atom, and an Al atom. Specific examples of the inorganic amphoteric ion exchanger having the combination of a Zr atom, a Mg atom, and an Al atom include IXEPLAS-A1 and IXEPLAS-A2 (both of which are trade names (manufactured by TOAGOSEI CO., LTD.). Note that “IXE” and “IXEPLAS” are registered trademarks).

In an adhesive according to the present invention, the form of the inorganic amphoteric ion exchanger is not particularly limited, may be, for example, particulate or amorphous, and is preferably particulate. The inorganic amphoteric ion exchanger has a median diameter of, for example, preferably 0.1 to 10 μm, more preferably 0.1 to 5 μm, still more preferably 0.1 to 3 μm.

The median diameter can be determined on the basis of “particle size analysis: laser diffraction methods” defined in JIS Z8825-1:2013, using a particle size distribution measurement apparatus (for example, LA-950V2 (trade name) manufactured by HORIBA, Ltd.).

In an adhesive according to the present invention, the content of the inorganic amphoteric ion exchanger is not particularly limited and is, for example, relative to 100 parts by mass of the epoxy resin serving as Component (A), preferably 0.1 parts by mass or more and 20 parts by mass or less, more preferably 0.2 parts by mass or more and 15 parts by mass or less, more preferably 1 part by mass or more and 10 parts by mass or less, more preferably 2 parts by mass or more and 8 parts by mass or less, still more preferably 2.5 parts by mass or more and 7 parts by mass or less.

### Cured Product

A cured product according to the present invention is a cured product generated by curing an adhesive according to the present invention. Specifically, a cured product according to the present invention is used as a member forming a bonding portion in an endoscope. The curing temperature of an adhesive according to the present invention is not particularly limited. In an adhesive according to the present invention, the curing reaction efficiently proceeds even in a low-temperature region, to provide a cured product according to the present invention. The components are mixed together preferably while bubbles are removed, which is hence ordinarily performed under a reduced pressure. The curing temperature is preferably 100° C. or less, more preferably 80° C. or less, still more preferably 60° C. or less, or can be set at 50° C. or less. In order to sufficiently cause the curing reaction, the curing temperature is preferably 0° C. or more, more preferably 10° C. or more. The curing-reaction time can be appropriately set in accordance with the purpose. Ordinarily, the curing reaction is caused for 1.5 to 200 hours, to provide the cured product.

Endoscope

In an endoscope according to the present invention, a cured product according to the present invention is used to fix a constituent member. This phrase “a cured product according to the present invention is used to fix a constituent member” means that at least one of the constituent members of the endoscope is fixed to a support member using a cured product according to the present invention.

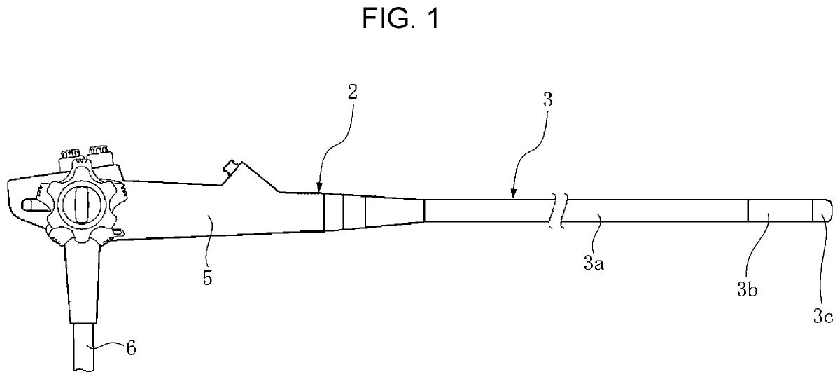

An example of an endoscope (electronic endoscope) according to the present invention will be described. The electronic endoscope includes therein an endoscopic flexible tube (hereafter, the endoscopic flexible tube may also be simply referred to as “flexible tube”), and is widely used as a medical device. In the example illustrated in FIG. 1, an electronic endoscope 2 includes an insertion section 3 to be inserted into the body cavity, a main-body operation section 5 coupled to the base-end portion of the insertion section 3, and a universal cord 6 connected to a processor device and a light source device. The insertion section 3 is constituted by a flexible tube 3a coupled to the main-body operation section 5, an angle portion 3b coupled to the flexible tube 3a, and a distal-end portion 3c coupled to the distal end of the angle portion 3b and mainly constituted by metal (such as stainless steel) members. This distal-end portion 3c houses an imaging device (not shown) for imaging the inside of the body cavity. The flexible tube 3a, which accounts for most of the length of the insertion section 3, has flexibility substantially over the whole length; in particular, the portion inserted into inner regions such as the body cavity has a more flexible structure.

In FIG. 1, from the main-body operation section 5 to the distal-end surface of the distal-end portion 3c, a plurality of channels (tubes, not shown) extending throughout in the axial direction of the insertion section 3 are formed.

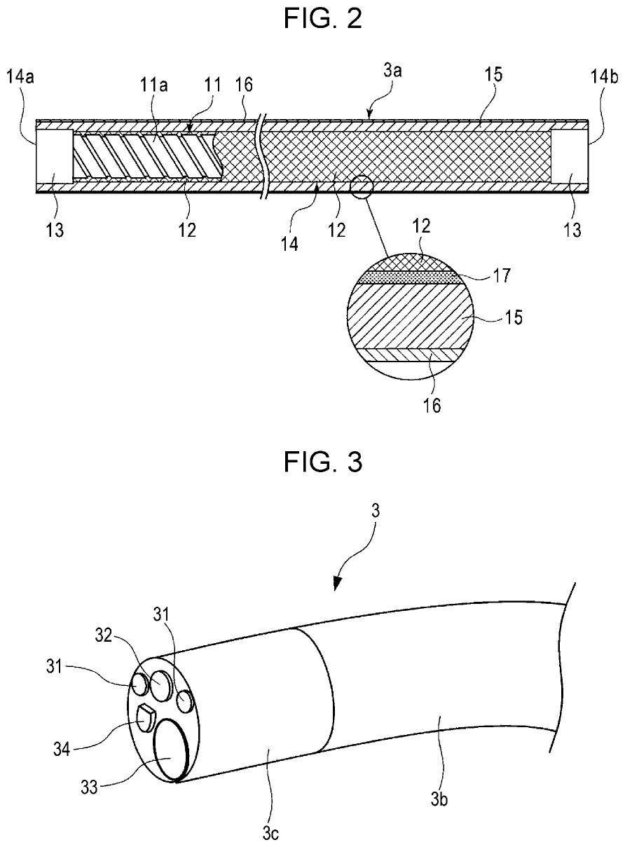

The flexible tube 3a in FIG. 1 has a configuration in which, as illustrated in FIG. 2, the outer peripheral surface of a flexible-tube base 14 is covered with a resin layer 15.

Reference sign 14a denotes the distal-end side (distal-end portion 3c side) while Reference sign 14b denotes the base-end side (main-body operation section 5 side).

The flexible-tube base 14 is formed by covering a spiral tube 11, which is disposed on the innermost side and formed by spirally winding a metal strip 11a, with a sleeve mesh body 12 formed by knitting metal wires. Both ends of the flexible-tube base 14 are fitted with metal caps 13. This resin layer 15 is bonded to the flexible-tube base 14 with an adhesive-cured-product layer 17 therebetween. This adhesive-cured-product layer 17 can be formed by applying and curing an adhesive according to the present invention. The adhesive-cured-product layer (bonding portion) 17 is drawn as a uniform and thick layer for the purpose of illustration, but does not necessarily have this form, and may be disposed in an amorphous form between the resin layer 15 and the flexible-tube base 14. Alternatively, the layer may barely have a thickness and the resin layer 15 and the flexible-tube base 14 may be bonded together substantially in contact with each other.

The outer surface of the resin layer 15 is coated with a coating layer 16 having chemical resistance and containing, for example, fluorine. Incidentally, the adhesive-cured-product layer 17, the resin layer 15, and the coating layer 16 are drawn at large thicknesses relative to the diameter of the flexible-tube base 14 for the purpose of clearly illustrating the layer structure.

As illustrated in FIG. 3, at the distal-end surface of the distal-end portion 3c, illumination windows 31, an observation window 32, and a forceps port 33 are formed. In addition, in order to wash the distal-end surface as needed, a nozzle 34 for sending out water and the air is formed. The illumination windows 31, the observation window 32, the forceps port 33, and the nozzle 34 are coupled, via channels, to the main-body operation section 5.

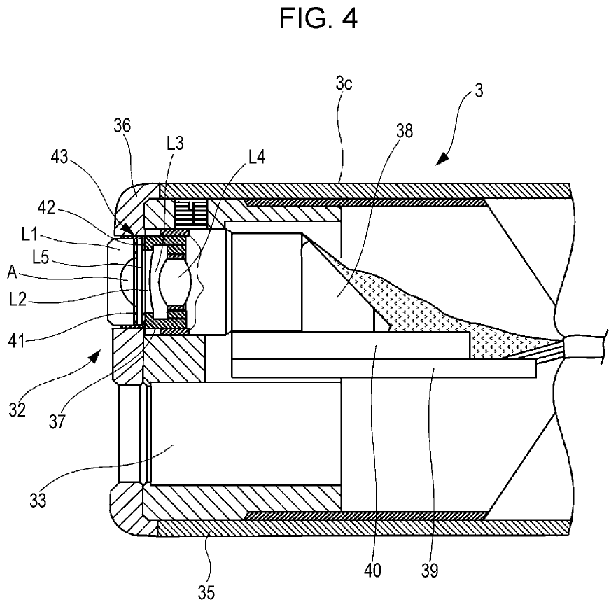

As illustrated in FIG. 4, the distal-end portion 3c is constituted by a distal-end-portion main body 35 formed of metal, and a distal-end cap 36 formed of an electrically insulating member.

At the observation window 32, an observation unit 43, which is an optical device, is disposed. In the observation unit 43, within a lens holder 37, an objective optical system constituted by lenses L1 to L5 is fixed using adhesive cured products 41 and 42. These adhesive cured products 41 and 42 can be formed by applying and curing an adhesive according to the present invention. In this objective optical system, Reference sign A denotes an air layer. To an end surface of the lens holder 37, a prism 38 is bonded and fixed. This prism 38 perpendicularly deflects the optical axis of the objective optical system. This prism 38 is fixed to a solid image pickup element 40. The solid image pickup element 40 is fixed to a substrate 39. These can be fixed also with an adhesive according to the present invention.

### Method for Producing Endoscope

A method for producing an endoscope according to the present invention is not particularly limited as long as it includes using an adhesive according to the present invention to fix an endoscope constituent member; as the steps other than the fixing of the endoscope constituent member, ordinary production steps can be employed to produce an endoscope according to the present invention.

The endoscope constituent member to be fixed is not particularly limited in terms of material, and examples include resin members, metal members, and glass members. The endoscope constituent member can be fixed to a support member or the like of an endoscope in the following manner: for example, the components included in an adhesive according to the present invention are mixed together preferably under a reduced pressure; subsequently, the mixture is injected or applied to the application point, and heated, for example, at −10 to 60° C. (preferably 0 to 60° C., more preferably 10 to 50° C.) for 1.5 to 200 hours.

Hereinafter, usage forms of the adhesive in a method for producing an endoscope according to the present invention will be described with reference to specific examples; however, the present invention is not limited to these.

Of endoscope constituent members fixed using an adhesive according to the present invention, a resin member is, for example, a tube inserted through the insertion section of the endoscope. Examples of the resin material forming the tube include fluororesins such as TEFLON (registered trademark), polysulfone, polyesters, polyolefins, and silicone. An adhesive according to the present invention can be used for, for example, bonding between a metal member or a glass member of the insertion section of the endoscope and the tube (fixing of the metal member or the glass member to the tube).

In addition, as described above, the adhesive can also be used to form the adhesive-cured-product layer 17 in FIG. 2. In addition, the adhesive can also be used to bond together, in FIG. 2, the resin layer 15 and the coating layer 16.

An adhesive according to the present invention can be used for outer-surface finishing and fixing of an end portion (distal-end side (angle portion 3b side) end portion of the flexible tube 3a) of the flexible outer cover tube (resin layer 15). Specifically, the end portion of the resin layer 15 of the flexible tube 3a is externally bound tightly using a thread and fixed to the internal member, and subsequently the adhesive is applied so as to cover the thread and cured. The outermost layer of the distal-end-side end portion of the flexible tube 3a is thus formed from an adhesive according to the present invention, so that the thread in this distal-end-side end portion becomes less likely to fray, and the insertion section is easily inserted into the body cavity. The insertion section formed in this manner can maintain its glossy appearance even after sterilization.

In addition, an adhesive according to the present invention can be used for at least one bonding of bonding between the distal-end portion 3c and the angle portion 3b or bonding between the insertion section 3 and the main-body operation section 5. For example, the distal-end portion 3c and the angle portion 3b are bonded together using an adhesive according to the present invention; subsequently, the region at and near the bonding portion between the distal-end portion 3c and the angle portion 3b is bound tightly using a thread to reinforce the bonding, and the adhesive is applied so as to cover the thread, and cured. The same applies to bonding between the insertion section 3 and the main-body operation section 5.

In addition, an adhesive according to the present invention can also be used for fixing of various tubes inserted through the insertion section of the endoscope, onto at least one of the distal-end portion 3c or the main-body operation section 5.

In addition, an adhesive according to the present invention is also preferably used, in the distal-end portion 3c, for sealing of the illumination windows 31 and the observation window 32 (fixing of the glass members). The adhesive can be applied at large thicknesses, to thereby smooth the peripheral corners of the lenses, and to block lateral entry of light into the lenses.

In addition, an adhesive according to the present invention can be used for fixing of members such as assembly of the imaging device housed within the distal-end portion 3c, bonding of parts, and sealing of the solid image pickup element 40. The imaging device has an optical system constituted by a plurality of optical parts such as the lenses L1 to L5 and the prism 38, and the solid image pickup element 40 that photoelectrically converts optical images formed by the optical system into imaging signals, such as a CCD (Charge Coupled Device). An adhesive according to the present invention can be used for, for example, bonding together of optical parts formed of a material such as glass that are the lenses L1 to L5, the prism 38, and the like, and bonding of the lenses L1 to L5, the prism 38, and the like to the substrate 39 formed of resin or metal; this bonding achieves fixing of the glass members, and fixing of the metal members.

In addition, an adhesive according to the present invention can be used for bond-fixing and sealing between the solid image pickup element 40 and the substrate 39. This bonding achieves fixing of the metal members constituting the solid image pickup element, the substrate, and the like.

Thus, a method for producing an endoscope according to the present invention includes a step of using an adhesive according to the present invention to fix an endoscope constituent member.

### EXAMPLES

The present invention will be described further in detail with reference to Examples; however, the present invention is not construed as being limited to Examples below. The term “room temperature” means 25° C. The formulation amount of each component means the formulation amount of the component itself. Specifically, when the raw material includes a solvent, the formulation amount does not include the amount of the solvent.

### Preparation Examples: Preparation of Adhesives

(A) an epoxy resin, (B) a curing component, and (C) an inorganic amphoteric ion exchanger were mixed together in amounts described in Table 1 below; such mixtures under stirring with an “AWATORI-RENTARO ARV-310 (trade name, manufactured by THINKY CORPORATION)” at room temperature, at a reduced pressure of 1.0 Pa, and at 2000 rpm, were defoamed for 5 minutes, to provide adhesives. Note that, in Test examples below, adhesives immediately after preparation were used.

Note that, in Comparative Examples 3 and 4, instead of the (B) curing component, X-1 described later was used. In Comparative Example 5, instead of the (B) curing component, X-2 described later was used. In Comparative Examples 6 to 8, instead of the (C) inorganic amphoteric ion exchanger, Y-1 to Y-3 described later were used.

### Test Examples

### Initial Curability (Low-Temperature Curability) Test

Such an adhesive (10 mL) obtained in Preparation examples above was poured into a 100 mL polypropylene disposable cup (manufactured by AS ONE Corporation), and left at rest at 30° C. Subsequently, the disposable cup was tilted and whether or not the adhesive flowed was visually determined. The time elapsed until the adhesive no longer flowed even during tilting of the disposable cup was defined as curing time, and graded into one of the following evaluation grades to evaluate initial curability. S, A, and B are pass grades of this test.

### Evaluation Grades of Initial Curability

S: a curing time of less than 6 hours

A: a curing time of 6 hours or more and less than 10 hours

B: a curing time of 10 hours or more and less than 20 hours

C: a curing time of 20 hours or more and less than 40 hours

D: a curing time of 40 hours or more

The results will be described in Table 1 below.

### Sterilization Treatment Durability (EOG (Ethylene Oxide Gas) Durability) Test

Such an adhesive obtained in Preparation examples above was cured using MINI TEST PRESS (manufactured by Toyo Seiki Seisaku-sho, Ltd.) at 50° C. for 24 hours. Thus, two sheet-shaped samples of 100 mm length×100 mm width×0.4 mm thickness (cured products) were obtained in each of Examples and Comparative Examples.

In an EOG sterilization apparatus (trade name “EQ-70”, manufactured by MIURA CO., LTD.), the high-temperature sterilization course (temperature: 55° C., relative humidity: 60%) was used to subject such a sheet-shaped sample to EOG sterilization treatment 50 times.

In each of Examples and Comparative Examples, one of the two sheets was defined as Sheet-shaped sample (I). The other sheet different from Sheet-shaped sample (I) was subjected to EOG sterilization treatment and defined as Sheet-shaped sample (II). Sheet-shaped samples (I) and (II) were subjected to a flexural test in accordance with JIS K7171 (2016) at a test speed of 2 mm/min and with a span between specimen supports of 70 mm. The flexural test was performed under an environment at 23° C., using a table-top type precision universal testing machine AGS-X (trade name, manufactured by SHIMADZU CORPORATION).

The ratio (%) of the flexural strength (MPa) of Sheet-shaped sample (II) to the flexural strength (MPa) of Sheet-shaped sample (I) (100×flexural strength (MPa) of Sheet-shaped sample (II)/flexural strength (MPa) of Sheet-shaped sample (I)) was defined as the retention ratio of flexural strength (MPa), and graded into one of the following evaluation grades to evaluate sterilization treatment durability. S, A, and B are pass grades of this test.

### Evaluation Grades

S: a retention ratio of flexural strength of 90% or more

A: a retention ratio of flexural strength of 80% or more and less than 90%

B: a retention ratio of flexural strength of 70% or more and less than 80%

C: a retention ratio of flexural strength of 60% or more and less than 70%

D: a retention ratio of flexural strength of less than 60%

The results will be described in the following Table 1.

TABLE 1

| | | | | | (C) Inorganic | | | |
|---|---|---|---|---|---|---|---|---|
| | (A) Epoxy resin | | (B) Curing component | | amphoteric ion exchanger | | Low- | |
| | | Parts | | Parts | | Parts | temperature | EOG |
| | Type | by mass | Type | by mass | Type | by mass | curability | durability |
| Example 1 | A-1 | 100 | B-1 | 40 | C-1 | 5 | B | B |
| Example 2 | A-2 | 100 | B-1 | 40 | C-1 | 5 | B | B |
| Example 3 | A-3 | 100 | B-1 | 40 | C-1 | 5 | B | B |
| Example 4 | A-1 | 100 | B-2 | 70 | C-3 | 3 | S | S |
| Example 5 | A-2 | 100 | B-3 | 3 | C-3 | 3 | B | B |
| Example 6 | A-2 | 100 | B-4 | 50 | C-3 | 3 | S | S |
| Example 7 | A-2 | 100 | B-5 | 20 | C-3 | 3 | B | B |
| Example 8 | A-2 | 100 | B-4 | 50 | C-2 | 10 | A | A |
| Example 9 | A-2 | 100 | B-4 | 50 | C-4 | 1 | A | A |
| Example 10 | A-2 | 100 | B-4 | 50 | C-5 | 5 | S | S |
| Example 11 | A-2 | 100 | B-4 | 50 | C-6 | 4 | S | S |
| Example 12 | A-2 | 100 | B-4 | 50 | C-2 | 15 | B | A |
| Example 13 | A-2 | 100 | B-4 | 50 | C-2 | 0.5 | A | B |
| Comparative | A-2 | 100 | B-1 | 40 | None | 0 | D | C |
| Example 1 | | | | | | | | |
| Comparative | A-2 | 100 | B-4 | 50 | None | 0 | D | C |
| Example 2 | | | | | | | | |
| Comparative | A-2 | 100 | X-1 | 40 | None | 0 | D | C |
| Example 3 | | | | | | | | |
| Comparative | A-2 | 100 | X-1 | 40 | C-1 | 5 | C | C |
| Example 4 | | | | | | | | |
| Comparative | A-2 | 100 | X-2 | 40 | C-1 | 5 | D | D |
| Example 5 | | | | | | | | |
| Comparative | A-2 | 100 | B-1 | 40 | Y-1 | 5 | D | C |
| Example 6 | | | | | | | | |
| Comparative | A-2 | 100 | B-1 | 40 | Y-2 | 5 | D | C |
| Example 7 | | | | | | | | |
| Comparative | A-2 | 100 | B-1 | 40 | Y-3 | 5 | D | C |
| Example 8 | | | | | | | | |

In Table
(A) Epoxy resin
A-1: Bisphenol A diglycidyl ether (trade name: “jER 828”, manufactured by Mitsubishi Chemical Corporation, epoxy equivalent: 190)
A-2: Bisphenol F diglycidyl ether (trade name: “EPICLON 830”, manufactured by DIC Corporation, epoxy equivalent: 170)
A-3: Epoxy novolac resin (product number: 406775, manufactured by Sigma-Aldrich Corporation, epoxy equivalent: 170)
(B) Curing component
B-1: Dicyandiamide (manufactured by Tokyo Chemical Industry Co., Ltd.)
B-2: Pentaerythritol tetrakis(3-mercaptobutyrate) (trade name “Karenz MT (registered trademark) PE1”, manufactured by SHOWA DENKO K. K.)
B-3: Triphenylphosphine (manufactured by Tokyo Chemical Industry Co., Ltd.)
B-4: Polyetheramine D400 (trade name, manufactured by Mitsui Fine Chemicals, Inc.) (Exemplary compound B-2 above)
B-5: Phenol resin (trade name “HF-1M”, manufactured by Meiwa Plastic Industries, Ltd.)
(C) Inorganic amphoteric ion exchanger
C-1: IXE-600 (trade name, manufactured by TOAGOSEI CO., LTD.)
C-2: IXE-633 (trade name, manufactured by TOAGOSEI CO., LTD.) (C-1 and C-2 are each an inorganic compound containing an Sb atom and a Bi atom.)
C-3: IXE-6107 (trade name, manufactured by TOAGOSEI CO., LTD.)
C-4: IXE-6136 (trade name, manufactured by TOAGOSEI CO., LTD.) (C-3 and C-4 are each an inorganic compound containing a Zr atom and a Bi atom.)
C-5: IXEPLAS-A1 (trade name, manufactured by TOAGOSEI CO., LTD.)
C-6: IXEPLAS-A2 (trade name, manufactured by TOAGOSEI CO., LTD.) (C-5 and C-6 are each an inorganic compound containing a Mg atom, an Al atom, and a Zr atom.)
Other components
X-1: m-Xylylenediamine (manufactured by Tokyo Chemical Industry Co., Ltd.)
X-2: 2-Ethyl-4-methylimidazole (manufactured by Tokyo Chemical Industry Co., Ltd.)
Y-1: DOWEX (registered trademark) 50Wx2 (organic cation exchange resin manufactured by The Dow Chemical Company)
Y-2: DIAION WA30 (organic anion exchange resin manufactured by Mitsubishi Chemical Corporation)
Y-3: IXE-600 (trade name, manufactured by TOAGOSEI CO., LTD., Zr atom-containing inorganic anion exchanger)

In Table 1 above, in order to facilitate comparison between Examples and Comparative Examples, X-1 and X-2 are described in the column of (B) Curing component and Y-1, Y-2, and Y-3 are described in the column of (C) Inorganic amphoteric ion exchanger.

In the cases of, without using inorganic amphoteric ion exchangers, using, as curing components, dicyandiamide and a polyether-polyamine compound defined in the present invention to cure an epoxy resin, the low-temperature curability and the sterilization treatment durability were found to be insufficient (Comparative Examples 1 and 2).

Similarly, in the case of, without using inorganic amphoteric ion exchangers, using, as a curing component, m-xylylenediamine to cure an epoxy resin, the low-temperature curability and the sterilization treatment durability were found to be insufficient (Comparative Example 3).

In the cases of using, in the presence of an inorganic amphoteric ion exchanger, m-xylylenediamine or 2-ethyl-4-methylimidazole as a curing component to cure an epoxy resin, the low-temperature curability and the sterilization treatment durability were found to be insufficient (Comparative Examples 4 and 5).

In the cases of using, in the presence of an organic ion exchanger (organic cation exchange resin or organic anion exchange resin), as a curing component, dicyandiamide defined in the present invention to cure an epoxy resin, the low-temperature curability and the sterilization treatment durability were found to be insufficient (Comparative Examples 6 and 7).

In the cases of using, in the presence of an inorganic anion exchanger, as a curing component, dicyandiamide defined in the present invention to cure an epoxy resin, the low-temperature curability and the sterilization treatment durability were found to be insufficient (Comparative Example 8).

By contrast, Examples 1 to 13 have each demonstrated that an adhesive according to the present invention has high low-temperature curability, and the cured product of the adhesive has high sterilization treatment durability. In addition, for example, the results of Examples 4 and 6 have demonstrated that use of, as curing components, a polythiol compound and a polyether-polyamine compound enables higher-level improvements in low-temperature curability and sterilization treatment durability. Furthermore, Example 5 has demonstrated that, in the presence of an inorganic amphoteric ion exchanger, even in the case of using triarylphosphine, which alone is less likely to achieve curing of an epoxy resin, sufficient low-temperature curability can be provided.

The present invention has been described together with embodiments thereof; however, we do not intend to limit our invention in any minor portion of the descriptions unless otherwise specified; we believe that the invention is construed broadly without departing from the spirit and scope of the invention described in the attached claims.

### REFERENCE SIGNS LIST

2 electronic endoscope (endoscope)
3 insertion section
   3a flexible tube
   3b angle portion
   3c distal-end portion
5 main-body operation section
6 universal cord
11 spiral tube
   11a metal strip
12 sleeve mesh body
13 metal cap
14 flexible-tube base
   14a distal-end side
   14b base-end side
15 resin layer
16 coating layer
17 adhesive-cured-product layer
31 illumination window
32 observation window
33 forceps port
34 nozzle
35 distal-end-portion main body
35 distal-end cap
37 lens holder
38 prism
39 substrate
40 solid image pickup element
41 adhesive cured product
42 adhesive cured product
43 observation unit
A air layer
L1 to L5 lenses

What is claimed is:

1. An adhesive for an endoscope, the adhesive comprising:
(A) an epoxy resin including at least one epoxy resin of a bisphenol A epoxy resin, a bisphenol F epoxy resin, or a phenol novolac epoxy resin;
(B) a curing component including at least one of a phosphorus-containing compound, a polythiol compound, a dicyandiamide compound, a phenol compound, or a polyether-polyamine compound; and
(C) an inorganic amphoteric ion exchanger.

2. The adhesive for an endoscope according to claim 1, wherein the curing component includes at least one of a polythiol compound or a polyether-polyamine compound.

3. The adhesive for an endoscope according to claim 1, wherein the inorganic amphoteric ion exchanger is an inorganic compound including at least one species of a bismuth atom, an antimony atom, a zirconium atom, a magnesium atom, or an aluminum atom.

4. The adhesive for an endoscope according to claim 1, wherein a content of the inorganic amphoteric ion exchanger relative to 100 parts by mass of the epoxy resin is 1 part by mass or more and 10 parts by mass or less.

5. A cured product provided by curing the adhesive for an endoscope according to claim 1.

6. An endoscope comprising a member fixed using the cured product according to claim 5.

7. A method for producing an endoscope, the method comprising fixing a member using the adhesive for an endoscope according to claim 1.
